(19) United States
(12) Patent Application Publication
(10) Pub. No.: US 2014/0340639 A1
(43) Pub. Date: Nov. 20, 2014

(54) METHOD AND SYSTEM FOR DETERMINING THE RELATIVE GAZE-ATTRACTING POWER OF VISUAL STIMULI

(71) Applicant: Langbourne W. Rust, Philadephia, PA (US)

(72) Inventor: Langbourne W. Rust, Philadephia, PA (US)

(73) Assignee: Langbourne Rust Research Inc., Philadelphia, PA (US)

(21) Appl. No.: 14/271,451

(22) Filed: May 6, 2014

Related U.S. Application Data
(60) Provisional application No. 61/819,851, filed on May 6, 2013.

(51) Int. Cl. A61B 3/113

(52) U.S. Cl. CPC A61B 3/113
USPC 351/209; 351/246

(57) ABSTRACT

An system and method for enabling an individual to pretest stimulus alternatives, by carrying out eye-tracking experiments on a subject with a digital camera, a personal computer and webcam, or a camera built into a laptop, tablet, smart phone, or other mobile device. The method of implementing the invention may be based on use of the internet for transmitting experimental stimuli, data, and/or results. The method enables the practitioner to determine which of two or more competing stimulus images, displayed at the same time, draws more spontaneous gaze direction from the subject during the first few moments of exposure. The method enables one to express the result as a ratio or other comparative index.

### FIELD OF THE INVENTION

The invention is in the field of visual stimulus reaction measurement and analysis, in particular via the use of eye tracking.

### BACKGROUND OF THE INVENTION

A. Stimulus Testing.

The goal of stimulus testing is to produce stimulus scores that reflect the underlying, inherent ability of the stimulus itself to affect the interest of a person, without distortions from extraneous factors that can influence how a person reacts to the stimulus. A variety of stimulus pretesting systems are known, all of which have certain limitations and drawbacks. (American Marketing Association, New York Chapter (2012), Green Book: International directory of marketing research companies and services New York; Center for Substance Abuse Prevention (1994), Pretesting is essential: You can choose from various methods (Technical Assistance Bulletin), Washington, D.C., U.S. Government Printing Office; National Cancer Institute, National Institutes of Health, Making Health Communication Programs Work: Developing and Pretesting Concepts, Messages, and Materials (online at www.cancer.gov); Wells, W. D. (Ed.) (1997), Measuring advertising effectiveness. Mahwah, N.J., Lawrence Erlbaum Publishers.)

How a particular person reacts to a particular stimulus at a particular moment in time can be influenced by many things, such as the mode of presentation, the context in which the stimulus is displayed, and the physical and social setting in which the experience takes place. Other factors include the unique characteristics of the individual such as educational and cultural background, as well as the current emotional and physical state and recent thoughts or actions of the respondent. These extraneous influences are regarded as confounding factors, leading to errors in the estimate of the true value of the stimulus's capacity to elicit whatever response is being measured. A primary challenge of stimulus testing is to minimize the effects of these variables on the behavior of interest.

In practice, attempts are made to eliminate the influence of such extraneous factors on the scores, typically by randomization, by environmental standardization, or though statistical controls. Typical strategies include:

1) Careful sample matching, so that each stimulus has a similar sample;

2) A large sample size, so that the unique characteristics and conditions of individual subjects get averaged out in the aggregated scores;

3) Standardized protocols, so that all subjects are given the same instructions, given the same tasks, and asked the same questions in the same way;

4) A standardized display, so that all tested stimuli are shown in the same way, with the same surrounding stimuli, framing, medium, etc.;

5) Randomized presentation sequence, so that any effects arising from a stimulus being experienced early or late in a session are evenly distributed across the different stimuli; and

6) A standardized social and physical setting. Typically, individual subjects are isolated from others in order to reduce social setting differences. Central location testing is employed, so that all subjects use the same or similar devices under identical conditions, in the same or similar facilities.

A secondary challenge is predictive validity: The test must generate scores that are predictive of future responses of the population in question, in the settings of interest. Misrepresentative samples, atypical protocols, tasks, displays and settings, and atypical psychological and social conditions can interfere with predictive validity.

Finally, there is the challenge of relevance: The test must measure, and predict, behavior and responses that matter to the end-users of the research findings, using relevant measurement units, and avoid or give little weight to behaviors and responses that are not relevant to the end user.

Creators of stimuli (e.g., advertisements, product design, packaging, or entertainment) need to know what people will look at in everyday life. Today's over-crowded stimulus environments make it imperative that communicators and designers create materials that will cut through the clutter in people's visual fields. It is broadly understood that they need to “grab” people's eyes in the first few seconds of exposure. To do this, they need to know what people are inclined to look at spontaneously and unself-consciously, when they are comfortable, relaxed, and not engaged in an assigned task where results of some sort are expected. What people look at when they are uncomfortable and feeling self-conscious, in a strange environment, and expecting to be quizzed about what they see, is not likely to reflect their spontaneous reactions in everyday life. For this reason, current stimulus pre-testing systems do not measure what creative people need most to know.

Such systems typically seek to determine and measure what people think. Most stimulus-comparison measures use self-conscious, cognitively-mediated reports of people's opinions, thoughts, memories, intentions, preferences and emotions. These are collectively known as “reactive” measures. They do not give accurate reflections of the pre-conscious, instant behavioral reactions that people make to stimuli. While they may predict important aspects of people's reactions after they have focused on an image, and have had time to reflect on it and construct an opinion, reactive measures to an image do not predict whether subjects will focus on that image spontaneously in the first place, in a real-world setting. That spontaneous focus is a function of the unconscious, non-cognitive elements of perception, i.e., processes within the brain that take place before a subject has had a chance to “think about” what is being seen. Researchers studying human perception and cognition have found evidence of a time interval, the so-called “three-second window”, where human perception appears to operate optimally; see Pöppel, E. (2004) “Lost in time: a historical frame, elementary processing units and the 3-second window.” Acta Neurobiologiae Experimentalis, 64:295-301 and Schleidt, M. and Feldhütter, I. (1989) “Universal Time Constant Operating in Human Short-term Behaviour.” Naturwissenschaften, 76:127-128.)

Many systems measure how people react in unrepresentative settings. The few non-reactive measures in use today, such as eye-tracking and physiological or neurological monitoring, are highly intrusive, require elaborate instrumentation and laboratory settings, and lead to very artificial viewing experiences. Currently used methods and systems study longer-term and continuous reaction patterns, and do not focus on the initial, pre-cognition phase of human reactions to visual stimuli.

Most measure either how a stimulus scores against a broad set of other stimuli, or how one part of the stimulus scores against another part. Creative decision makers need information that focuses on the choice at hand, i.e., the “Which one is better” question. They are not so concerned with the “How good is it” question, which is what commercial copy testing services tend to address, by producing test scores that draw their meaning from a body of norms built from all the testing that has been done previously. Among the downsides of this approach are statistical issues related to sampling error.

Differences between stimulus-reaction scores obtained from separate samples of respondents can result not just from differences between the stimuli themselves, but also from differences (many of them uncharted) between the samples.

B. Eye Tracking.

As used herein, the term “eye tracking” refers broadly to methods of determining what a person is looking at by observing the position and orientation of the head and/or eyes. As used herein, the term encompasses methods variously referred to as eye tracking, gaze tracking, eye monitoring, eye position monitoring, and the like.

Eye-tracking and gaze-tracking systems and methods are known. See for example Hansen, D. W. and Ji, Q., 2010, “In the Eye of the Beholder: A Survey of Models for Eyes and Gaze.” IEEE Trans. Pattern Anal. Mach. Intell. 32(3): 478-500; Majaranta, P., Aoki, H., Donegan, M., Hansen, D. W., Hansen, J. P., Hyrskykari, A., and Räihä, K-J. (Eds.) 2011, Gaze Interaction and Applications of Eye Tracking: Advances in Assistive Technologies, IGI Global; Hammoud, R. I. (Ed.), 2008, Passive Eye Monitoring, Springer; Bojko, Aga, (2011), Eye Tracking: the User Experience. A Practical Guide, Rosenfeld Media, online at rosenfeldmedia.com; and Duchowski, A. T. (2002) “A Breadth-First Survey of Eye Tracking Applications,” Behavior Research Methods, Instruments, and Computers. 34(4):455-70. Commercial systems and software are readily available for the purpose, for example from The Eyetrack Shop, New York, N.Y.; The Pretesting Company, Tenafly, N.J.; and Tobii Technology, Danderyd, Sweden. Various limitations and drawbacks exist with these systems and methods.

Some eye-tracking methods provide continuous measures of eye movements, in order to track fixation points & paths within an image. The more elaborate systems require captive audiences, and employ intrusive technology and hardware such as specialized goggles, lasers, head-mounted cameras, and so forth. The methods may be combined with additional camera-derived information, such as blink rate, and data derived from physiological monitoring of the subjects' body, such as pulse rate and skin conductivity. Such sophisticated apparatus, in turn, requires a central laboratory, to which the subjects must travel to be tested. The facilities and their staff are expensive to maintain, and the subjects are placed in a highly unnatural environment with a correspondingly unnatural state of mind.

The Distractor Method is used to study aggregated attention flow scores to moving video materials such as TV programming, commercials, videos, and film. See Fisch, Shalom M.; Rosemarie T. Truglio (eds.) (2001), “G” is for Growing: Thirty Years of Research on Children and Sesame Street. Mahwah, N.J.: Lawrence Erlbaum Publishers; and Rust, L. (1987), “Using Attention and Intention to Predict In-Home Program Viewing”, J. Advert. Res., 272, 25-30.

The Distractor Method uses generalized or randomized distractor stimuli, typically videos or still images, to standardize distraction effects from one score and one test to another. The distractor method generates normative, generalizable data, which is typically used as a measure of how much attention a show or scene gets from an audience.

Reactive measurement systems solicit respondents' opinions, preferences, judgments, recognition, recall or other consciously-produced reactions. They require the test respondents to make cognitive judgments, to report or make other consciously-mediated actions. The demand for active, directed responses to stimuli has been shown to systematically alter peoples' perceptions and reactions to stimuli, which significantly degrades the ability of the test results to predict future behavior occurring under other conditions.

Physiological reactions such as brain wave, heart rate, galvanic skin response, etc. are also used to measure and evaluate reactions to stimuli, and to plot changes in those reactions over time. These methods require direct physical contact with the respondent, and usually require central location testing, highly specialized and intrusive measurement apparatus, and trained staff to operate and maintain the facility.

So-called A/B site testing services use an experimental design model to compare the real-world performance of alternate websites or pages, and they employ a variety of metrics (clickthroughs, navigation paths, visit duration, reactive self-reports from respondents, etc.). They require using different samples for different stimuli, and do not measure glance direction of respondents.

There is a need for behavior and response measurement methods that do not suffer from the disadvantages discussed above.

### BRIEF DESCRIPTION OF THE INVENTION

The prior art methods of “How good is it?” testing require large samples that are carefully screened to be as similar as possible; this, in turn, requires considerable time, effort and expense. The present invention provides “Which is better” testing, with stimulus scores coming from the same sample of people. This eliminates the problem of sampling error clouding the scores, and as a result it can be quicker, less expensive, more reliable and more sensitive than prior art methods. In particular, by testing visual stimuli, the system of the present invention measures what matters most to people who create visual content, images, and media.

As noted above, the distractor method generates normative, generalizable data, in an attempt to measure how much attention a given performance gets from an audience. The current invention, in contrast, employs a side-by-side comparison of two specific, still images, and the score of each image is dependent upon the gaze-attraction power of the other. The method accordingly generates non-normative scores, confining generalizations only to the two-image comparison.

Through the use of internet technology, the current invention allows data to be gathered from people while they are in their own homes, on their own computers, in a relaxed state of mind. The subjects are simply watching a changing display of graphic images, with no conscious “reactive” judgments being asked of them. The system of the invention unobtrusively acquires images of the subjects' faces, which are used to create a clear record of what they actually looked at. This record enables the practitioner of the method to track and analyze which individuals looked at which stimuli throughout the exposure period.

The present invention uses unsupervised respondents, in familiar settings with familiar hardware, passively watching images on a screen and not being asked to rate, judge, remember or think about them. The method differentiates only whether a subject looks toward one stimulus or another, rather than toward pieces, elements or sections of a larger image. The present invention differentiates the stimulus images based on the subject's spontaneous reactions during the first few seconds of exposure. This is accomplished by eye-tracking, which in turn is carried out unobtrusively via a digital camera, preferably a camera built into the personal device (computer, laptop, tablet, or mobile phone) on which the subject is watching the test images. The system notes which images are on the screen, where on the screen the images are, and, via eye tracking, which images have the attention of the subject, how quickly the attention was acquired, and for how long the attention is held. Analysis of this data according to the methods of the invention provides useful and relevant scores, which are directly related to the responses of the subjects to the images.

The present invention collects data within the first few seconds of exposure to the two stimulus images. Specifically, data on gaze direction is obtained within the first eight seconds of exposure, preferably within the first five seconds. More preferably, data is collected within the first three seconds of exposure. It is anticipated that the point of focus after a short time period may differ from the focus a few seconds later. Data obtained at different time points may be relevant to different real-world environments, and the collection, analysis, and use of such time-dependent information is within the scope of this invention. The results obtained by the methods of the present invention have the advantage of being more relevant to the problem of securing the attention of consumers in a real-world environment cluttered with competing visual stimuli, such as a supermarket aisle or a newspaper or magazine advertising page.

The system of the present invention harnesses the capabilities of the internet in a novel way to provide valid but affordable testing systems that address the immediate decision-making needs faced by creators, and answer precisely the questions that they ask every day: Which of two options (typically, images and/or text) should I go with? Which will more effectively cut through the clutter? Which will get us the most “eyeballs”?

The method of the invention provides a way to manage the three sets of challenges described above:

1) Between-stimulus sampling error is eliminated entirely by the nature of the research question (which image do they look at?), and by confining analyses to comparisons between paired images. The two stimuli are viewed at the same time by the same people, and their scores are not compared to data on other images, other times or other people.

2) Predictive validity is greatly enhanced by testing people in their own, everyday, familiar settings on their own familiar devices, by unobtrusively studying a behavior that is spontaneous, non-reactive and un-self-conscious, and by having the people perform a task that takes no thought and no effort on their part.

3) Relevance is provided by measuring stimulus units of direct and immediate interest to the end user (who wants to decide which image to go with), using response units that are relevant (respondent gaze direction in the first few moments of exposure) and behavior that is important (breaking through the clutter of competing images.)

### BRIEF DESCRIPTION OF THE FIGURES

FIG. 1 summarizes the overall process of testing images using this invention.

FIG. 2 shows two examples of image pairs suitable for testing under the method and system of the invention.

FIG. 3 shows an example of an image pair displayed on a personal digital computer or mobile device screen.

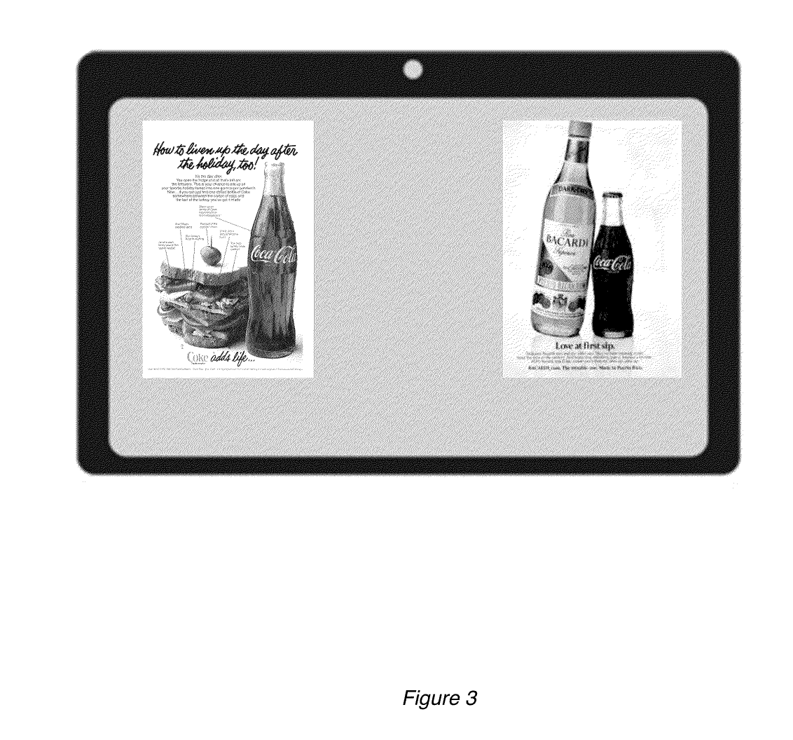

FIG. 4 gives two examples of webcam images from a stimulus pretest according to this invention.

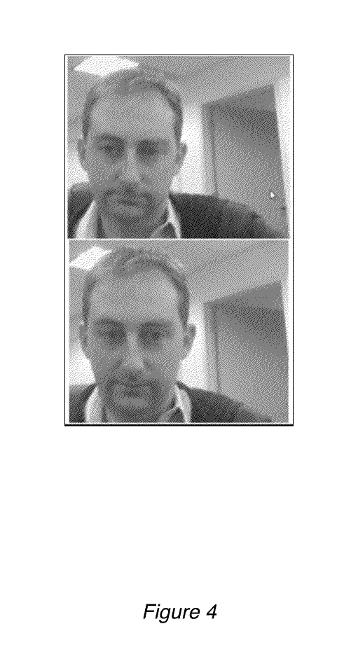

FIG. 5 details the steps involved in project design (01).

FIG. 6 shows the steps involved in the setup phase (02).

FIG. 7 shows the steps involved in a data collection session under the embodiment in which the webcam takes periodic snapshots of the respondent during stimulus display.

FIG. 8 shows the steps involved in a data collection session when the webcam is producing a continuous video of the respondent during stimulus display.

FIG. 9 shows the steps involved in processing the webcam data.

FIG. 10 lists some of the possible forms of output that can be generated by the invention.

FIG. 11 is a schematic of key components and information flow for a data collection session.

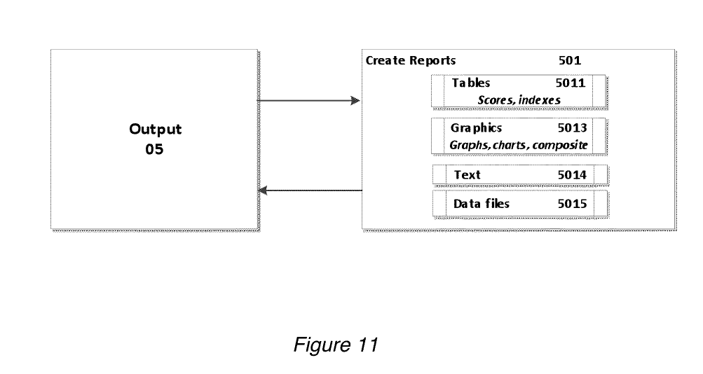

FIG. 12 is a graph of the comparative second-by-second full-sample gaze-scores of two images, in a stimulus pair exposed for 3 seconds.

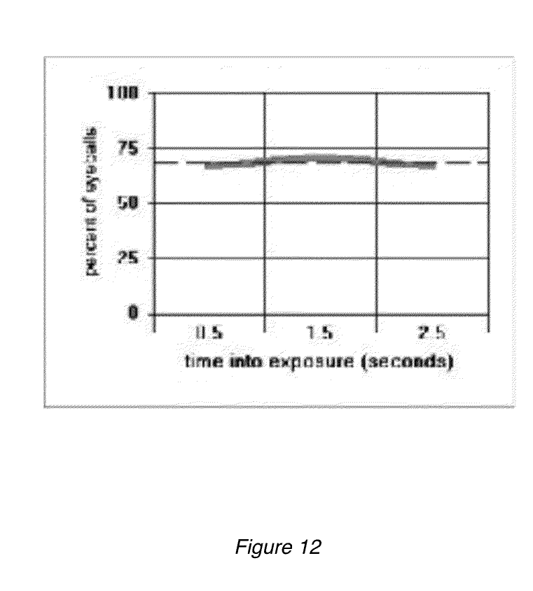

### DETAILED DESCRIPTION OF THE INVENTION

Broadly, the invention provides a computer-implemented method of determining the response of an individual subject to a pair of visual stimuli, which includes the steps of:

a. Causing a first image and a second image (the visual stimuli) to simultaneously appear on the screen of a display device;

b. Causing a camera attached to or built in to the display device to record the direction of gaze of the subject over a pre-determined period of time;

c. Causing said display device to transmit to a receiving computer the recorded direction of gaze, the identity of the images displayed at the time of the recording, and the location on the screen of each of the images; and

d. Calculating how often the subject was coded as gazing towards the first image and how often the subject was coded as gazing towards the second image, during the first few seconds of exposure.

In certain embodiments, the first image and the second image may be transmitted from a server to the display device. The display device itself may be a personal display device, in the possession of the subject, or it may be part of or attached to the server. The receiving computer may be incorporated into (i.e., built into) the display device, or, in other embodiments, it may be the server or a computer networked to the server. Examples of display devices include but are not limited to computer monitors operatively linked to the server or to a personal computer in the possession of the subject, and the display screens of smart phones, PDAs, or laptop, notebook, or tablet computers.

In preferred embodiments, the images are transmitted from a server to a personal display device that is in the possession of the subject, so that the subject can be exposed to the visual stimuli while in a natural and familiar environment such as the subject's home or office. The camera may be a separate digital still or video camera, such as a webcam, but it is preferably a camera built into the subject's personal computer display, laptop, notebook, tablet, or smart phone.

Images of the subject, showing the direction of the subject's gaze, may be transmitted to the server as they are obtained, or they may be accumulated and stored in the personal device for later transfer to the server. The images of the subject are coded to indicate which of the visual stimuli the subject was gazing at at the time the image was obtained, and the data is further processed as indicated below to determine which of each pair of stimuli attracted the greater amount of attention, and to determine the relative proportion of attention was given to each of the images in each pair. Additional pairwise presentations are made, until the desired number of paired stimuli have been viewed by the subject.

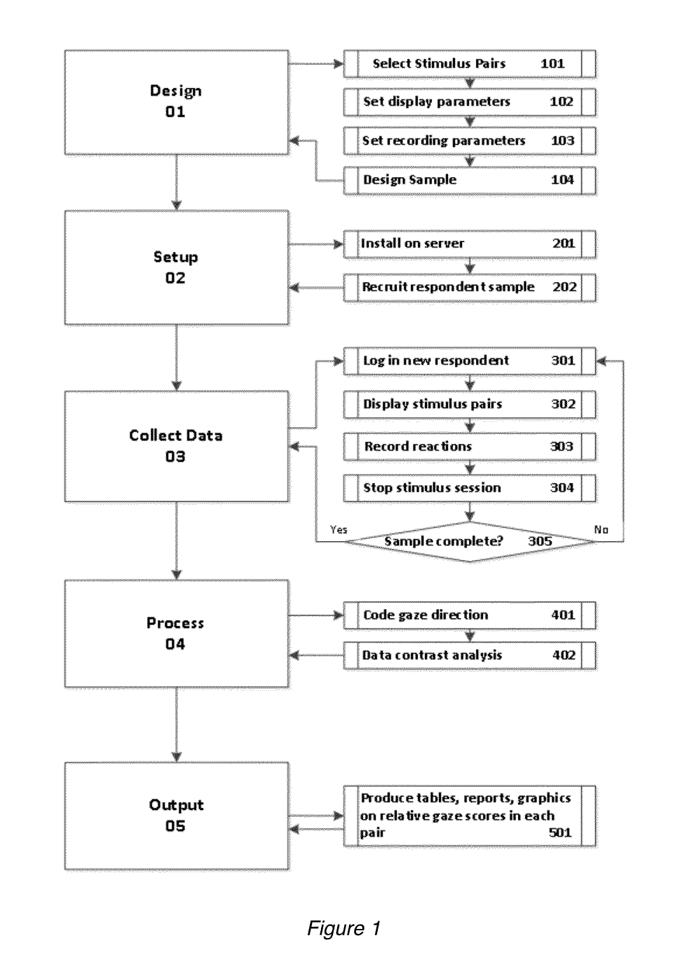

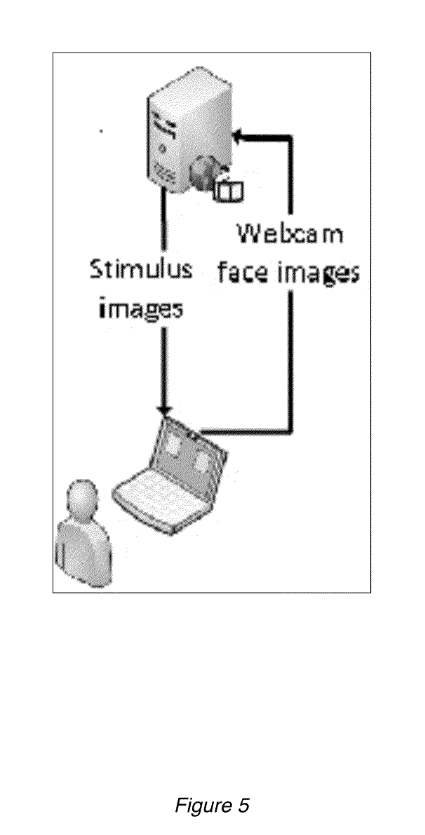

As shown in FIG. 1, stimulus-pair testing begins with the design phase (01) in which the visual images to be tested are identified (101), the display and recoding parameters are set (102 and 103), and the sample is designed (104). The sub-steps that are required in each of the design steps are set out in FIG. 5.

In the particular embodiment shown, defining the stimuli (101) involves locating the image file (1011); validating the adequacy of the located file in terms of file type (for browser-readability), file size and content (1012); assigning each stimulus to a partner stimulus, to which it is to be compared (1013); and saving these specifications to a project template file (1014). In alternative embodiments, the specifications can be hard-coded into a testing app, to enable the display of these images during data collection (03).

Setting the display parameters (102) involves defining how long the image pairs are to be exposed (1021); the properties of the two frames in which the images are to be displayed (e.g., their size, shape and location on the display screen) (1022); and the appearance of the display screen such as background color and pattern (1023). The display specifications may be saved to a template file for reference, or put into software code directly.

Setting the recording parameters (103) involves setting the recording mode for either still (snapshot) or video webcam recoding (1031); the compression type and ratio to be employed by the webcam-driving and image-receiving software (1032); and the timing parameters. In the case of snapshot mode, timing parameters include when and at what intervals snapshots are taken during the exposure of a stimulus pair, and in the case of video mode, they include when to start the video recording and when to stop it (1033).

Designing the sample (104) involves defining which respondents will qualify for testing, using variables such as demographics, ownership and usage of digital devices and webcams, and other criteria appropriate to the goals and intentions of the current test (1041). Sample design may also includes setting the desired sample and subgroup sizes (1042), writing a screening interview to identify qualifying respondents (1043), and storing the specifications in some form, such as a template file, to structure the administration of the sample recruitment process (1044).

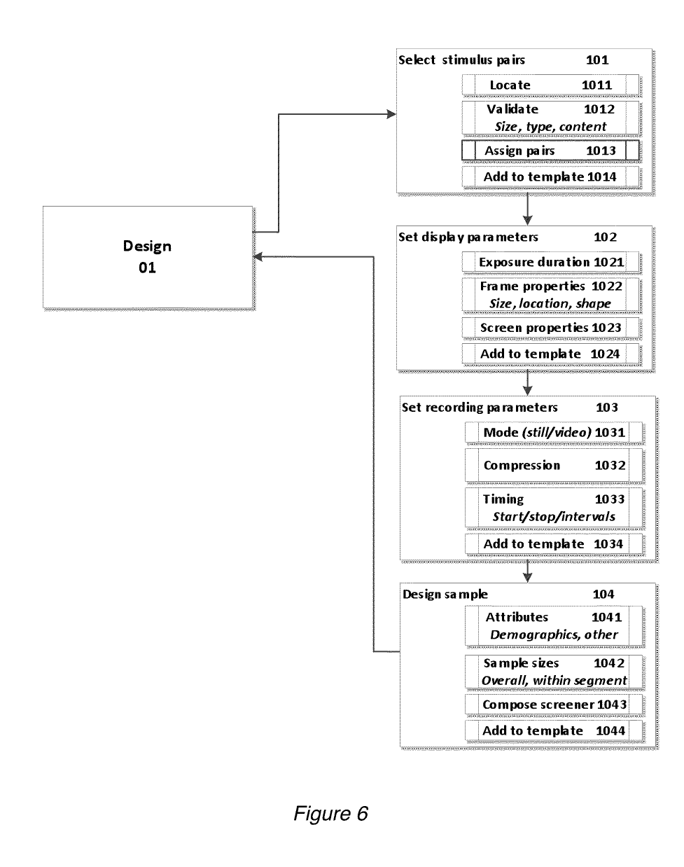

FIG. 6 outlines a typical setup process for conducting data collection (02). This involves the installation on the server (201) of the stimulus files (2011), and the study templates governing their display and recording and respondent interactions (2012)

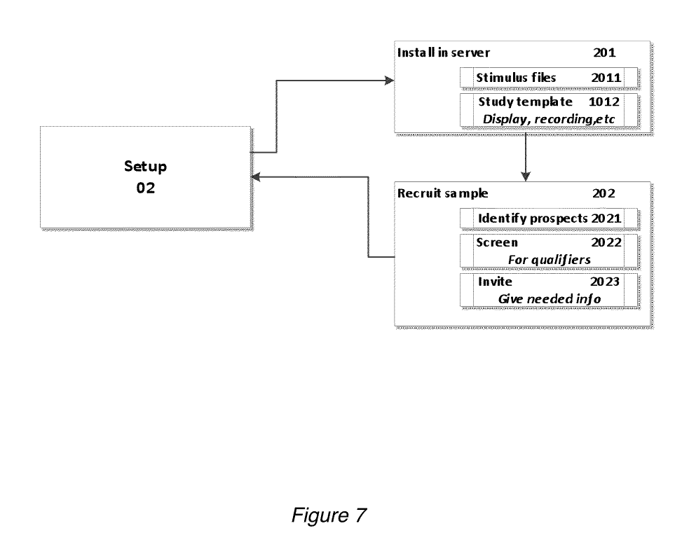

FIG. 7 outlines the data collection process (03) in the embodiment in which data collection uses the webcam to take snapshots of the respondent during the stimulus display session. A respondent logs in to the test site from their personal device (301). The server-side software validates the respondent as qualified for the survey (3011), has them check the respondent's system and webcam to make sure they produce a readable picture is (3012); and creates a randomized, respondent-unique rotation and position schedule for displaying stimulus pairs, if more than one stimulus pair is being tested (3013).

During the stimulus display session (302), an image pair is displayed on the respondent's device (3021) and the webcam is triggered to record reactions (303).

The reaction recording process (303) involves taking snapshots at predefined intervals (3031), making a log entry which includes a timestamp, the snapshot filename, and the filenames and positions of the two images currently on display (3032). Snapshots are acquired and uploaded to the test server (3033) until the last snapshot scheduled for the stimulus pair exposure has been taken (3034)

When the stimulus pair display period has ended (3022), if it is not the last pair scheduled, another pair is displayed (3021) and the process is repeated. If it is the last scheduled pair, the viewing session is ended (304), the session log file is uploaded (3041), and the camera is turned off (3042).

Respondents continue to be tested until the sample quota, as set by the sample specifications (3013) has been reached (305).

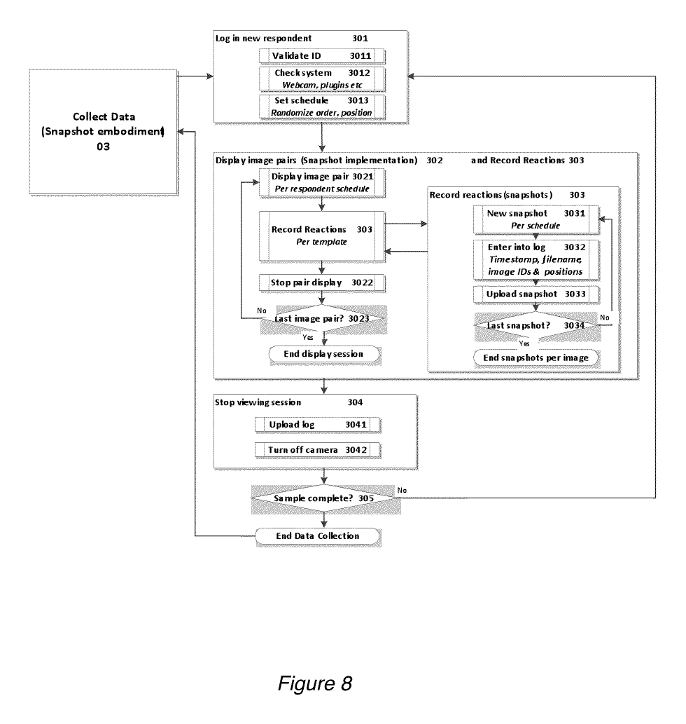

FIG. 8 outlines the analogous data collection process (03) when the webcam takes continuous video rather than snapshots. A respondent logs in to the test site from their personal device (301). The server-side software validates the respondent as qualified for the survey (3011), has them check the respondent's system and webcam to make sure they produce a readable picture (3012); and creates a randomized, respondent-unique rotation and position schedule for displaying stimulus pairs, if more than one stimulus pair is being tested (3013).

During the exposure session with video recoding, recording begins first (306). Once the camera is running (3061), the image display session is started (307). Image pairs are displayed, according to the respondent's randomized schedule (3013). An entry is made into the session log (3072) containing a timestamp and the filenames and positions of the stimuli being displayed. After the specified display period, the stimulus images are removed (3073) and, optionally, another entry is made in the session logfile (3074).

Images are displayed, and recordings of the subject are taken, for a period of time that is sufficient for the desired calculations. The calculations are made based on recordings obtained during about the first eight seconds of exposure of the subject to the stimulus images, preferably during the first five seconds. Recordings obtained during other time periods, for example at 2, 4, or 6 seconds after the initial exposure, may optionally be employed at the discretion of the practitioner. The portion of the recordings that is utilized may be that beginning at the moment of initial exposure, or beginning after a brief initial exposure period has passed. Suitable initial exposure periods include, but are not limited to, 0.1, 0.2, 0.3, 0.4, or 0.5 seconds.

At the end of the last image pair display period (3075), the image display session ends (307) and the recording session (3063) ends. When the viewing session is over (304), the session log is uploaded to the server (3041) and the camera is turned off (3042).

Here also, respondents continue to be tested until the sample quota, as set by the sample specifications (3013) has been reached (305).

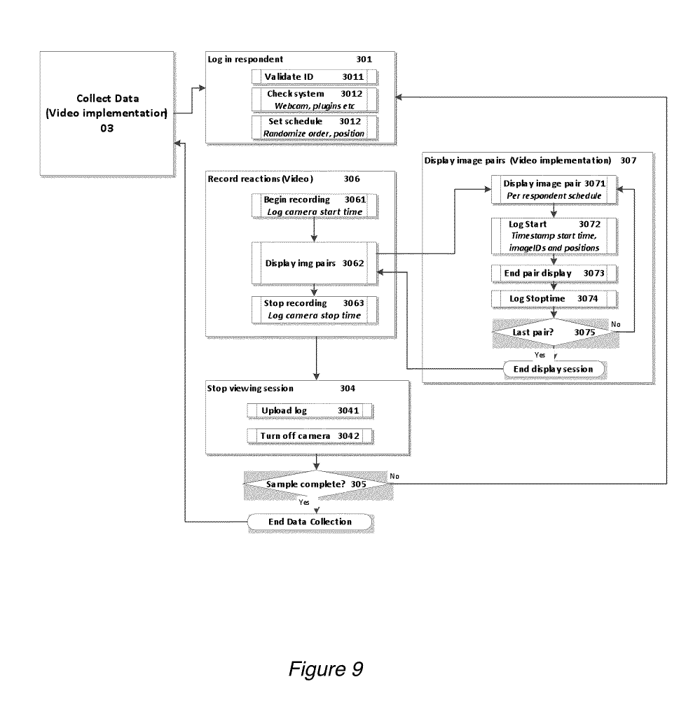

FIG. 9 outlines representative data processing procedures (04). Gaze direction is coded from the webcam image files of the respondent faces (401). For each respondent, a still image is selected for coding, either one of the snapshots, under snapshot mode recording, or a still-frame image from the video under video mode. Respondent gaze is coded as directed towards one stimulus or the other, or not coded at all in the event of shut eyes, unclear image, etc (4012). The coding may be done by a human, who views the images and assigns a gaze direction based on visual evaluation, and/or by software which carries out the same function via image analysis.

Reference to the session log file identifies the stimuli being displayed at the time of the image, their positions on screen and their timing within the image pair exposure period. The codes, the respondent identifier and the coded-image log-data are stored to a database or other file (4014) for later analysis. This continues until the last image is coded (4015).

Analysis of the attention code data (402) proceeds with aggregation of the stored codes (4021). Numerous aggregations are possible, but the key ones are the aggregation (tabulation) of Boolean code scores (looking towards/not) for each stimulus in a stimulus-pair;

1) over time—across all the coded time points within that exposure over time (e.g. “Image X was looked at by respondent A for 2 of the 3 moments that were coded, while Image Y was looked at once”); and

2) over respondents—across all respondents who were exposed to that stimulus. It can be aggregated across respondents (e.g., “At 0.5 seconds into the XY pair display, 80 respondents were looking at X, 20 were looking at Y”) and an aggregation across both time and respondents (e.g., “Image P got 220 positive attention codes, overall, versus Item Q's 80”.)

Analysis is performed on the aggregated data, contrasting the differences in gaze-attraction frequencies (4022) between the images within each pair. From the frequency tabulations, analysis progresses to the calculation of scores, derivation of statistics and generation of relative-performance indices (4023) to characterize the gaze-attracting differences between the competing images. Examples of relative-performance indices include, but are not limited to percent-of-all-gaze codes, e.g., “Image X got 75 percent of all of attention coded for the XY pair”, calculated as Pₓ=100*(Fₓ/Fₜₒₜ) where P=percent, Fₓ=Frequency of gaze codings to stimulus X, Fₜₒₜ=Frequency of all gaze codings to either X or Y; and a ratio or “lift” index based on these calculations, e.g., “Image X got looked at 90% more than Y”, calculated as Lₓ=Pₓ/P_{y}−100 where Lₓ=Lift score of stimulus X, and Pₓ and P_{y} are the percent scores of X and Y as calculated by the percent-of-all-gaze scores formula.

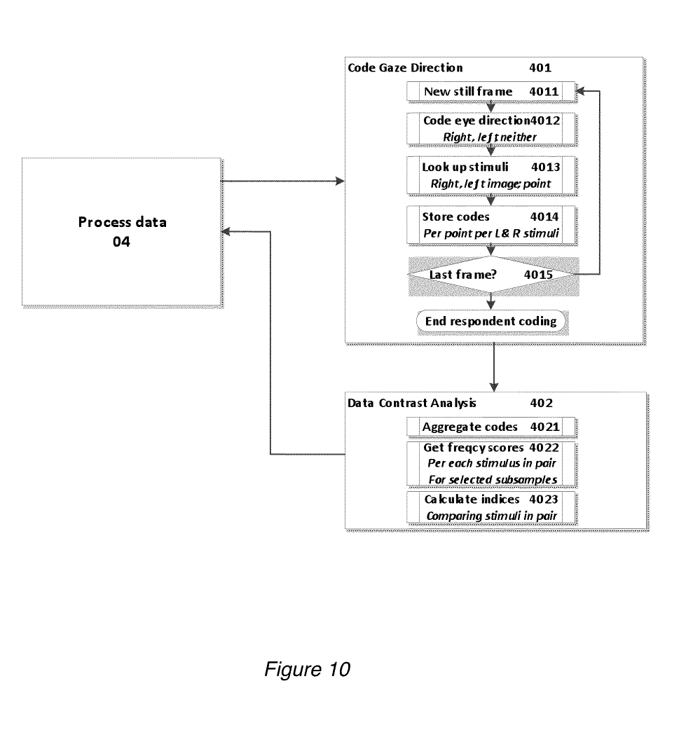

FIG. 10 lists some of the possible forms of output (05) that can be generated by the data processing. These include the generation of reports which can include tables (5011) of frequencies, indices and statistics, graphics (5012) such as charts, graphs or composite images overlaying images with numbers or graphic representations of the relative scores (5013), descriptive or analytical text (5014) and digital data files or recordset tables for export.

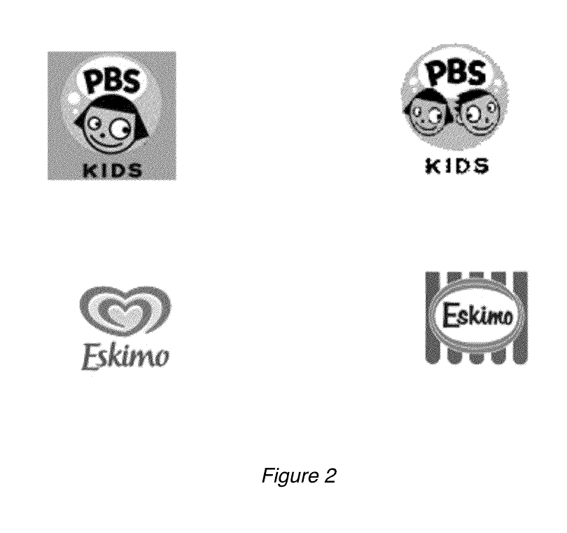

Table 1 is a table of percent-of pair's gaze scores for stimulus scores with chi-square and confidence statistics. This is actual data obtained by the methods of the invention; the catalog pictures and corporate logos are those shown in FIG. 2.

TABLE 1

| | Pct. of | | Pct. of | Chi | |
|---|---|---|---|---|---|
| Stimulus | eyes | Partner | eyes | Square | Confidence |
| Catalog picture X | 65 | Catalog picture Y | 35 | 18.4 | .999 |
| Corporate logo 1982 | 58 | Corporate logo 2010 | 42 | 5.8 | .98 |
| Politician's face, stern | 52 | Politician's face, smiling | 48 | 0.19 | 0 |

Table 2 is an example of relative-lift indexes for tested stimulus pairs, calculated from gaze scores, where the lift index expresses how many more positive attention codes (eyeballs) the winner got than the loser.

TABLE 2

| | | | Lift | |
|---|---|---|---|---|
| Stimulus | | Partner | Score | Confidence |
| Catalog picture X | versus | Catalog picture Y | 86% | .999 |
| Corporate logo 1982 | versus | Corporate logo | 38% | .98 |
| | | 2010 | | |
| Politician's face, stern | versus | Politician's face, | 8% | 0 |
| | | smiling | | |

The system components include both server-side components, for controlling image display, response recording, data management and analysis, and client-side components on the respondents' computer device.

Server-side components may include the digital files for each pair of stimulus images to be shown to respondents on personal devices; file management routines for the stimulus image files, for the webcam pictures of viewers' faces, for synchronizing files, and naming or otherwise identifying data sent back from respondent's device; and project template files. Also included may be browser instructions to control image display, response-image-collection, display-by-response synchronizing and recordkeeping and data processing software to code respondent gaze-direction from the facial-response images, to aggregate, tabulate and statistically analyze the data and output software to produce and configure the reports and export the data. The data-processing software could physically reside anywhere, either on the server or on some other system; in preferred embodiments it is one of the service components.

Client-side components include a digital camera or webcam attached to or built into the respondent's device, and a browser capable of playing images from a server on screen, per server instructions, while simultaneously controlling the client's webcam. These are capabilities currently conferrable by browsers offering HTML5 support, or which can support, for example, Microsoft SILVERLIGHT™, Apple QUICKTIME™ or Adobe FLASH™ plugins.

A preferred method of implementing the invention is via a syndicated data service business (see U.S. Pat. No. 7,272,582, which is incorporated herein by reference in its entirety.) The invention can support a service business in which a research service supplier conducts a single study for multiple clients, each submitting a pair of images for testing and getting back the relative gaze-scores for each. Pilot testing with the system has shown that respondents are comfortable viewing up to 30 different pairs of images in one sitting (a 90 second session), with no sign of fatigue and no indication that the scores of images seen near the start differ systematically from those seen near the end. This design is made possible by the fact that the non-reactive viewing experience is a natural, relaxed and passive one, which is very close to the subjects' everyday viewing experience of looking at things on their own device. The costs of the testing can be spread across all the participating clients, and the fees charged to each one of them is reduced correspondingly.

The invention can also support a custom service business, in which a customer submits all the stimulus items to be tested and the service provider conducts the test for them. The custom service model is particularly appropriate for customers who have many stimuli to test, who have unique sample-recruitment requirements, who want unique questioning and probing to follow up the exposure session, or who need to keep their research explorations better hidden from potential competitors.

The system software can be distributed under license to parties who want to conduct their own testing, for cost, customizability, confidentiality, control or other reasons, or to offer the service as a business themselves.

No other method uses webcam and internet technology to combine natural viewing environments, passive viewing conditions, and short-term paired image displays to unobtrusively measure people's spontaneous gaze direction between images shown in pairs.

I claim:

1. A computer-implemented method of determining the response of an individual subject to a pair of visual stimuli, comprising the steps of:
a. Causing a first image and a second image to simultaneously appear on the screen of a display device;
b. Causing a camera attached to or built in to the display device to record the direction of gaze of the subject over a pre-determined period of time;
c. Causing said display device to transmit to a receiving computer the recorded direction of gaze, the identity of the images displayed at the time of the recording, and the location on the screen of each of the images; and
d. Calculating how often the subject was coded as gazing towards the first image and how often the subject was coded as gazing towards the second image.

2. The method of claim 1, wherein the first image and the second image are transmitted from a server to the display device.

3. The method of claim 2, wherein the display device is a personal display device in the possession of the subject.

4. The method of any one of claims 2-3, wherein the receiving computer is the server.

5. The method of any one of claims 1-3, wherein the display device is operatively linked to, or built into, the receiving computer.

6. The method of any one of claims 2-3, wherein the display device is operatively linked to, or built into, the server.

7. The method of any one of claims 1-3, wherein the images are presented at a time t=0, and the step of calculating how often the subject was coded as gazing towards the first image and how often the subject was coded as gazing towards the second image is limited to a time period where 0<t<8 seconds.

8. The method of claim 7, wherein the time period is 0<t<5 seconds.

9. The method of claim 8, wherein the time period is 0<t<3 seconds.

10. The method of claim 9, wherein the time period is 0.5<t<3 seconds.
